(19) United States
(12) Patent Application Publication
(10) Pub. No.: US 2022/0012315 A1
(43) Pub. Date: Jan. 13, 2022

(54) AUTHORIZATION SYSTEM BASED ON BIOMETRIC IDENTIFICATION AND METHOD THEREFOR

(71) Applicant: NATIONAL TAIWAN UNIVERSITY, Taipei (TW)

(72) Inventors: An-Yeu WU, Taipei (TW); Ting-Wei SUN, Taipei (TW)

(21) Appl. No.: 16/922,005

(22) Filed: Jul. 7, 2020

(51) Int. Cl. G06F 21/32; A61B 5/00; A61B 5/0402

(52) U.S. Cl. CPC G06F 21/32; A61B 5/0402; A61B 5/7267; A61B 5/726

(57) ABSTRACT

An authorization system based on biometric identification and a method thereof are provided. An incomplete physiological signal of a subject is obtained. Next, the incomplete physiological signal is analyzed according to a machine learning model to identify an identity corresponding to the subject, and then output identity information. Then, whether the authorization is obtained based on the identity information is determined. When the authorization is obtained, the authorization content is provided. Therefore, in the case where the physiological signal is an incomplete signal, it is possible to perform identity recognition based on the machine learning model, and then to determine whether to provide the corresponding authorization content, so as to achieve the technical efficacy of recognition stability.

### BACKGROUND

### 1. Technical Field

The present invention relates to an authorization system and a method thereof. In particular, the invention pertains to an authorization system based on biometric identification and a method thereof.

### 2. Related Art

Existing common authorization method is that after a user inputs a password such as a combination of the numbers, the text and the symbols into a device through a keyboard, a mouse, a touchpad on a touch screen or other input devices, the input password must pass the verification process to make the user obtain authorization to start the device or execute the corresponding program in the device. However, there is the process of entering the password in the above method, so it is vulnerable to prying eyes to generate the problem of password leakage.

In order to solve the above problem, some companies propose to use the physiological signal, such as heartbeat, fingerprint, palm print, eye iris, and sound, to perform authorization determination, but there is a problem that when the physiological signal, which is with complete physiological characteristics, cannot be obtained due to the design of the sensing device, the environment, improper operation of the user, etc., the physiological signal needs to be reconstructed before identification can be performed, otherwise the identity recognition fails due to the physiological signal, which is without complete physiological characteristics, and the authorization cannot be obtained.

In summary, it can be seen that there is a problem that when the physiological signal, which is with complete physiological characteristics, cannot be obtained due to the design of the sensing device, the environment, improper operation of the user, etc., the physiological signal needs to be reconstructed before identification can be performed, otherwise the identity recognition fails due to the physiological signal, which is without complete physiological characteristics, and the authorization cannot be obtained. Therefore, it is necessary to propose an improved technical solution to solve this problem.

### SUMMARY

In order to solve the problem of the prior art, the present invention discloses an authorization system based on biometric identification and a method thereof.

First, the present invention discloses the authorization system based on biometric identification, which includes a sensing module, an identification module and an authorization module, wherein the identification module is connected to the sensing module, and the authorization module is connected to the identification module. The sensing module is configured to obtain a first physiological signal of a subject, wherein the first physiological signal is an incomplete signal; the identification module is configured to analyze the first physiological signal according to a machine learning model to identify an identity corresponding to the subject, and then output identity information; and the authorization module is configured to determine whether an authorization is obtained based on the identity information, and provide authorization content when the authorization is obtained.

In addition, the present invention discloses the authorization method based on biometric identification, which including the following steps: obtaining a first physiological signal of a subject, wherein the first physiological signal is an incomplete signal; analyzing the first physiological signal according to a machine learning model to identify an identity corresponding to the subject, and then output the identity information; and determining whether an authorization is obtained based on the identity information, and providing authorization content when the authorization is obtained

The system and method disclosed in the present invention are as above, and the difference from the prior art is that obtaining the incomplete physiological signal; analyzing the incomplete physiological signal according to the machine learning model to identify the identity corresponding to the subject, and then output identity information; determining whether the authorization is obtained based on the identity information; and providing the authorization content when the authorization being obtained.

By the above technical means, the present invention can perform identity recognition based on the machine learning model under the condition that the physiological signal is the incomplete signal, and then determine whether to provide the corresponding authorization content to achieve the technical effect of good stability of the recognition.

### BRIEF DESCRIPTION OF THE DRAWINGS

The structure, operating principle and effects of the present invention will be described in detail by way of various embodiments which are illustrated in the accompanying drawings.

FIG. 1A is a schematic diagram of components of an embodiment of an authorization system based on biometric identification of the present invention.

FIG. 1B is a system architecture diagram of an embodiment of the authorization system based on biometric identification of the present invention.

FIG. 2 is a flowchart of an embodiment of an authorization method based on biometric identification executed by the authorization system based on biometric identification of FIG. 1.

### DETAILED DESCRIPTION

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise”, “include” and “have”, and variations such as “comprises”, “comprising”, “includes”, “including”, “has” and “having” will be acknowledged to imply the inclusion of stated elements but not the exclusion of any other elements.

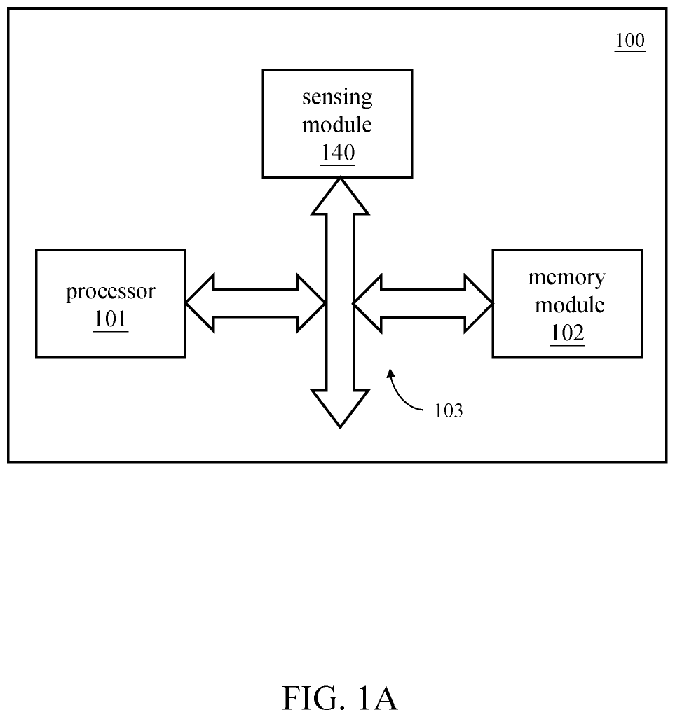

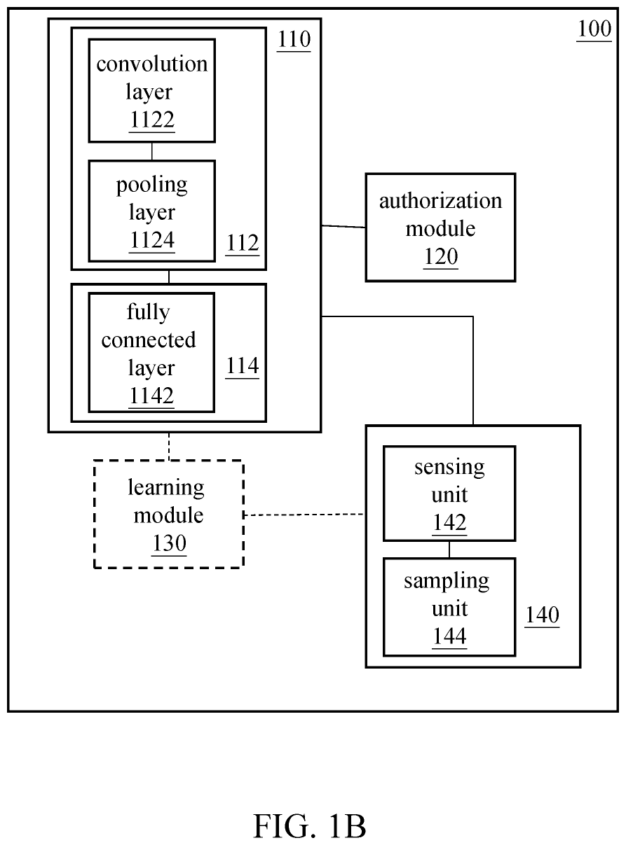

Please refer to FIG. 1A and FIG. 1B, wherein FIG. 1A is a schematic diagram of components of an embodiment of an authorization system based on biometric identification of the present invention, FIG. 1B is a system architecture diagram of an embodiment of the authorization system based on biometric identification of the present invention. In this embodiment, the authorization system based on biometric identification 100 may include but not limited to one or more processors 101, one or more memory modules 102, buses 103, a sensor (i.e., a sensing module 140) and other hardware components, wherein the buses 103 can be connected to different hardware components. Through the included multiple hardware components, the authorization system based on biometric identification 100 can be applied to an electronic device to execute software or applications.

The buses 103 may include one or more types of buses. For example, the buses 103 may include data buses, address buses, control buses, expansion buses, local buses and other types of buses. The buses of the electronic device include, but are not limited to, parallel industrial standard architecture (ISA) buses, peripheral component interconnect (PCI) buses, video electronics standards association (VESA) local buses, universal serial buses (USB), fast peripheral component interconnect express (PCI-E) buses, etc.

In this embodiment, the processor 101 may be coupled to the buses 103. The processor 101 may include a register group or register space. The register group or register space may be completely disposed on the processing chip, or may be disposed entirely or partially outside the processing chip and coupled to the processor 101 via the dedicated electrical connections and/or via the buses 103. The processor 101 may be a processing unit, a microprocessor, or any suitable processing element. When the authorization system based on biometric identification 100 includes multiple processors, the processors may be the same or similar processors, and are coupled to each other and communicate with each other through the buses 103. The processor 101 can interpret a series of multiple instructions to perform specific operations, such as mathematical operations, logical operations, data comparison, and copying/moving data, to execute various applications, modules, and/or components.

In addition, the processor 101 may be coupled to a chipset or electrically connected to the chipset through the buses 103, wherein the chipset is composed of one or more integrated circuits (IC), including a memory controller and a peripheral I/O controller. That is to say, the memory controller and the peripheral I/O controller can be included in one integrated circuit, or can also be implemented using two or more integrated circuits. The chipset usually provides the I/O and memory management functions, and provides multiple general-purpose and/or dedicated registers, timers, etc., wherein the above-mentioned general-purpose and/or dedicated registers and timers can be accessed or used by the one or more processors 101 coupled or electrically connected to the chipset.

Moreover, the processor 101 may also access data in the memory module 102 and the mass storage area installed on the authorization system based on biometric identification 100 through the memory controller. The above memory module 102 includes any type of volatile memory and/or non-volatile memory (NVRAM), such as a static random access memory (SRAM), a dynamic random access memory (DRAM), a flash memory, and a read-only memory (ROM). The above-mentioned mass storage area may include any type of storage device or storage medium, such as a hard disk, an optical disc, a flash memory, a memory card, a solid state disk (SSD) and any other storage device. In other words, the memory controller can access data in the SRAM, the DRAM, the flash memory, the hard disk, and the SSD.

Furthermore, the processor 101 can also connect and communicate with peripheral devices or interfaces, such as peripheral output devices, peripheral input devices, communication interfaces, and GPS receivers, via the buses 103 through the peripheral input/output controller. The peripheral input device can be any type of input device, such as a keyboard, a mouse, a trackball, a touchpad, a joystick, and a sensor (i.e., sensing module 140). The peripheral output device can be any type of output device, such as a display, and a printer, and the peripheral input device and the peripheral output device may also be the same device, such as a touch screen. The communication interfaces may include wireless communication interfaces and/or wired communication interfaces. The wireless communication interfaces may include a wireless local area network that supports Wi-Fi, Zigbee, etc., Bluetooth, infrared, near field communication (NFC), 3G/4G/5G and other mobile communication networks, or the interfaces for other wireless data transmission protocols. The wired communication interface can be an Ethernet device, an asynchronous transmission mode (ATM) device, a DSL modem, a cable modem, etc. The processor 101 may periodically poll various peripheral devices and interfaces, so that the authorization system based on biometric identification 100 can input and output data through the various peripheral devices and interfaces.

As shown in FIG. 1B, the authorization system based on biometric identification 100 includes a sensing module 140, an identification module 110, an authorization module 120, and an additional learning module 130. The identification module 110, the authorization module 120, and the learning module 130 are usually generated after the processor 101 executes a specific program loaded into the memory module 102, or included in the processor 101. In actual implementation, the authorization system based on biometric identification 100 can be applied but not limited to a smart phone, a tablet computer, a desktop computer, or a laptop.

In this embodiment, the sensing module 140 can be configured to obtain a first physiological signal of a subject, wherein the first physiological signal is an incomplete signal. Specifically, the incomplete signal is a physiological signal that does not have complete physiological characteristics. That is, the first physiological signal includes only a part of the physiological characteristics of the subject. For example, the first physiological signal is a partial fingerprint image, which only includes partial fingerprint features, the incomplete electrocardiogram signal (i.e., the incomplete ECG signal), which only includes some heartbeat features, or a partial facial image, which only includes some facial features. The reason why the sensing module 140 obtains the physiological signal, which is without complete physiological characteristics, may be, but not limited to, the design of the sensing module 140, the operating environment, and improper user operations. In actual implementation, the physiological characteristics included in the first physiological signal may be but not limited to heartbeat features or fingerprint features. That is, the first physiological signal may be the incomplete ECG signal or the incomplete fingerprint image. In other words, the first physiological signal may be a one-dimensional signal or a two-dimensional signal.

In this embodiment, the reason why the sensing module 140 obtains the physiological signal, which is without complete physiological characteristics, may be the design of the sensing module 140. In more detail, the sensing module 140 may include a sensing unit 142 and a sampling unit 144, and the sensing unit 142 is connected to the sampling unit 144. The sensing unit 142 can be configured to sense an actual physiological signal of the subject (i.e., the physiological signal, which is with complete physiological characteristics), such as the actual complete fingerprint image or the actual complete ECG signal. The sampling unit 144 can be configured to utilize a discrete cosine transform (DCT) technology, a discrete wavelet transformation (DWT) technology, a principal component analysis (PCA) technology, a compressive sensing (CS) technology or random sampling to process the actual physiological signal to generate the first physiological signal. In other words, the first physiological signal may be a compressed physiological signal or a randomly sampled physiological signal. In this embodiment, the physiological signal that does not have complete physiological characteristics is obtained through the design of the sensing module 140, which can reduce the size of the signal transmitted to the processor 101 and reduce the size of the signal subsequently processed by the processor 101. In addition, the physiological signal that does not have complete physiological characteristics can also retain the availability of the actual physiological signal and remove related information that violates privacy.

In this embodiment, the identification module 110 is connected to the sensing module 140, and can be configured to analyze the first physiological signal according to a machine learning model to identify an identity corresponding to the subject and output identity information. The machine learning model may be provided by the learning module 130 (the learning module 130 is connected to the identification module 110), or may be provided by a device other than the authorization system based on biometric identification 100. In addition, the machine learning model may be the module obtained by using the learning module 130 or the device other than the authorization system based on biometric identification 100 to train implementations of machine learning algorithms on a plurality of second physiological signals. Since the identification module 110 is based on the machine learning model to identify the identity of the subject, the machine learning algorithms used by the device other than the authorization system based on biometric identification 100 or the learning module 130 are the same as the machine learning algorithms used by the identification module 110 to perform the identification of the subject. The above-mentioned second physiological signals are complete physiological signals or incomplete physiological signals of multiple testers. When the above-mentioned machine learning model is provided by the learning module 130, the learning module 130 may obtain incomplete physiological signals of the testers (that is, the second physiological signals, which are incomplete signals) by the sensing module 140.

Since the machine learning model is a model obtained by using the learning module 130 to train implementations of machine learning algorithms on the complete physiological signals or incomplete physiological signals of the testers, when the identification module 110 identifies the identity corresponding to the subject, it determines which tester the subject is based on the machine learning model, and then outputs the corresponding identity information according to the determination result. Therefore, when one of the testers is the subject, the identification module 110 can accurately identify the identity corresponding to the subject.

The above machine learning algorithms may include quite a large number of different types of algorithms including implementations of a supervised learning algorithm, a unsupervised learning algorithm, a semi-supervised learning algorithm or a reinforcement learning algorithm. Specifically, the machine learning algorithms can include implementations of one or more of the following algorithms: a support vector machine, a decision tree, a nearest neighbor algorithm, a random forest, a ridge regression, a Lasso algorithm, a k-means clustering algorithm, a spectral clustering algorithm, a mean shift clustering algorithm, a non-negative matrix factorization algorithm, an elastic net algorithm, a Bayesian classifier algorithm, a random sampling consistency (RANSAC) algorithm, an orthogonal matching pursuit algorithm, a least squares regression and a convolutional neural network (CNN).

In this embodiment, the identification module 110 may include a feature extraction unit 112 and a classification unit 114. The feature extraction unit 112 is connected to the classification unit 114. The feature extraction unit 112 can be configured to perform feature extraction on the first physiological signal to obtain the feature values. The classification unit 114 can be configured to receive the feature values, and classify the feature values according to the machine learning model to identify the identity corresponding to the subject, and then output the identity information.

The identification module 110 may identify the identity corresponding to the subject based on the CNN algorithm (i.e., the machine learning algorithm used to obtains the above-mentioned machine learning model by the learning module 130 is the CNN algorithm), and then output identity information. In more detail, the feature extraction unit 112 may include a convolution layer 1122 and a pooling layer 1124, and the classification unit 114 may include a fully connected layer 1142, wherein the convolution layer 1122 is connected to the pooling layer 1124, and the pooling layer 1124 is connected to the fully connected layer 1142. The convolutional layer 1122 can be configured to perform feature extraction on the first physiological signal to obtain the multi-dimensional feature array. The pooling layer 1124 can be configured to reduce dimension of the multi-dimensional feature array to generate feature values. The fully connected layer 1142 can be configured to obtain a classification corresponding to the feature values according to the machine learning model, to obtain the identity corresponding to the subject, and then output the identity information, but this embodiment is not intended to limit the invention. For example, the feature extraction unit 112 may include multiple convolutional layers and multiple pooling layers, and the classification unit 114 may also include multiple fully connected layers. The convolutional layers and the pooling layers can be staggered and connected to each other. The convolutional layers and the pooling layers can be configured to generate lower-dimensional feature values, and the fully connected layers can be configured to classify and identify the feature values to identify the identity corresponding to the subject and output the identity information.

In this embodiment, the authorization module 120 is connected to the identification module 110, and can be configured to determine whether an authorization is obtained based on the identity information. When the authorization is obtained, the authorization content is provided. The method for determining whether the authorization is obtained is to determine whether the identity information is the same as the default identity information that can be verified. When the authorization is obtained, the authorization content provided by the authorization module 120 may be, but not limited to, starting the electronic device using the authorization system based on biometric identification 100 or executing the corresponding application in the electronic device using the authorization system based on biometric identification 100.

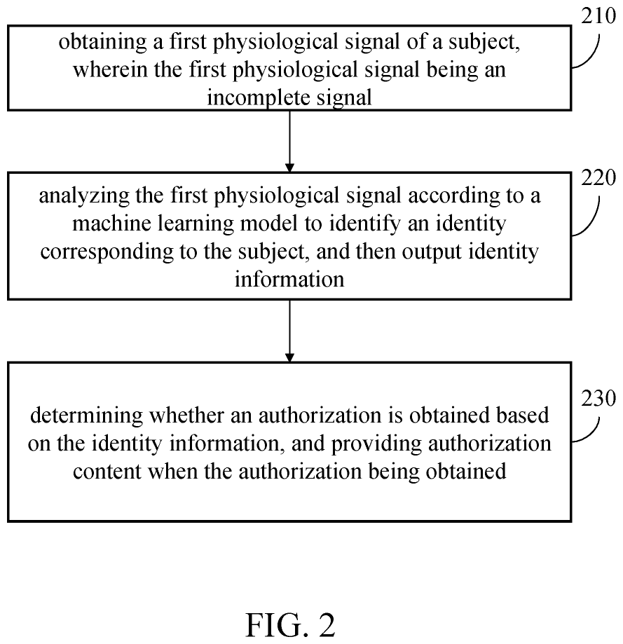

Next, please refer to FIG. 2, which is a flowchart of an embodiment of an authorization method based on biometric identification executed by the authorization system based on biometric identification of FIG. 1. The authorization method based on biometric identification may include the following steps: obtaining a first physiological signal of a subject, wherein the first physiological signal being an incomplete signal (step 210); analyzing the first physiological signal according to a machine learning model to identify an identity corresponding to the subject, and then output identity information (step 220); and determining whether an authorization is obtained based on the identity information, and providing authorization content when the authorization being obtained (step 230). The detailed description has been described above and is not repeated here.

Through the above steps, when the physiological signal is an incomplete signal, the identity can be identified based on the machine learning model, and then whether to provide the corresponding authorization content can be determined to achieve the technical effect of good stability of the identification.

In this embodiment, the step 210 may further include: sensing an actual physiological signal of the subject; and processing the actual physiological signal using a DCT technology, a DWT technology, a PCA technology, a CS technology, or random sampling to generate the first physiological signal. The detailed description has been described above and is not repeated here.

In this embodiment, the step 220 may further include: performing feature extraction on the first physiological signal to obtain feature values; and classifying the feature values according to the machine learning model to identify the identity corresponding to the subject, and then output the identity information. The detailed description has been described above and is not repeated here.

The step of performing feature extraction on the first physiological signal to obtain the feature values may further include: performing feature extraction on the first physiological signal to obtain a multi-dimensional feature array; and reducing dimension of the multi-dimensional feature array to generate the feature values. The detailed description has been described above and is not repeated here.

In this embodiment, the authorization method based on biometric identification can further include the following step: training implementations of machine learning algorithms on second physiological signals of multiple testers to establish the machine learning model based on a supervised learning algorithm, an unsupervised learning algorithm, a semi-supervised learning algorithm or a reinforcement learning algorithm, wherein each second physiological signal is an incomplete signal. The detailed description has been described above and is not repeated here.

In summary, the difference between the present invention and the prior art is that obtaining the incomplete physiological signal; analyzing the incomplete physiological signal according to the machine learning model to identify the identity corresponding to the subject, and then output identity information; determining whether the authorization is obtained based on the identity information; and providing the corresponding authorization content when the authorization being obtained, to achieve the technical effect of good stability of the recognition.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. An authorization system based on biometric identification comprising:
a sensing module configured to obtain a first physiological signal of a subject, wherein the first physiological signal being an incomplete signal;
an identification module connected to the sensing module and configured to analyze the first physiological signal according to a machine learning model to identify an identity corresponding to the subject and then output identity information; and
an authorization module connected to the identification module and configured to determine whether an authorization is obtained based on the identity information and provide authorization content when the authorization is obtained.

2. The authorization system based on biometric identification according to claim 1, wherein the sensing module includes a sensing unit and a sampling unit, the sensing unit is connected to the sampling unit, the sensing unit is configured to sense an actual physiological signal of the subject, and the sampling unit is configured to utilize a discrete cosine transform (DCT) technology, a discrete wavelet transformation (DWT) technology, a principal component analysis (PCA) technology, a compressive sensing (CS) technology or random sampling to process the actual physiological signal to generate the first physiological signal.

3. The authorization system based on biometric identification according to claim 1, wherein the authorization system based on biometric identification further comprises a learning module, which is connected to the identification module and the sensing module, and the learning module is configured to training implementations of machine learning algorithms on second physiological signals of multiple testers to establish the machine learning model based on a supervised learning algorithm, an unsupervised learning algorithm, a semi-supervised learning algorithm or a reinforcement learning algorithm, wherein each of the second physiological signals is an incomplete signal.

4. The authorization system based on biometric identification according to claim 1, wherein the identification module includes a feature extraction unit and a classification unit, the feature extraction unit is connected to the classification unit, the feature extraction unit is configured to perform feature extraction on the first physiological signal to obtain feature values, and the classification unit is configured to receive the feature values and classify the feature values according to the machine learning model to identify the identity corresponding to the subject, and then output the identity information.

5. The authorization system based on biometric identification according to claim 4, wherein the feature extraction unit includes a convolution layer and a pooling layer, the convolution layer is connected to the pooling layer, the convolution layer is configured to perform feature extraction on the first physiological signal to obtain a multi-dimensional feature array, and the pooling layer is configured to reduce dimension of the multi-dimensional feature array to generate the feature values.

6. An authorization method based on biometric identification, which comprising the following steps:
(a) obtaining a first physiological signal of a subject, wherein the first physiological signal being an incomplete signal;
(b) analyzing the first physiological signal according to a machine learning model to identify an identity corresponding to the subject, and then output identity information; and
(c) determining whether an authorization is obtained based on the identity information, and providing authorization content when the authorization being obtained.

7. The authorization method based on biometric identification according to claim 6, wherein the step (a) further comprising:
sensing an actual physiological signal of the subject; and
using a DCT technology, a DWT technology, a PCA technology, a CS technology or random sampling to process the actual physiological signal to generate the first physiological signal.

8. The authorization method based on biometric identification according to claim 6, wherein the authorization method based on biometric identification further comprises the following step:
training implementations of machine learning algorithms on second physiological signals of multiple testers to establish the machine learning model based on a supervised learning algorithm, an unsupervised learning algorithm, a semi-supervised learning algorithm or a reinforcement learning algorithm, wherein each of the second physiological signals being an incomplete signal.

9. The authorization method based on biometric identification according to claim 6, wherein step (b) further comprising:
(b1) performing feature extraction on the first physiological signal to obtain feature values; and
(b2) classifying the feature values according to the machine learning model to identify the identity corresponding to the subject, and then output the identity information.

10. The authorization method based on biometric identification according to claim 9, wherein step (b1) further comprising:
performing feature extraction on the first physiological signal to obtain a multi-dimensional feature array; and
reducing dimension of the multi-dimensional feature array to generate the feature values.
